SEQUENCE LISTING (16 sequences)

SEQ ID NO: 1 - DNA, 426 bp, Artificial Sequence
  It is synthesized.
gtgcagctgcaggagtctgggggaggctcggtgcaggtcggagggtctctgagactctcctgtgcctcctctgcaaacccctacagcagctactcactgggctggttccgccaggctccgggtaaggagcgcgagggagtcgcggctattaatggttatggtaggacaagttacgcagactccgtgagcggccgattcaccatctcccaagacaacgcccggaagactctgtatctgcaaatgaacagcctgaaacctgaggacactgccatgtactactgtgcaattgggtcgaggtggtggtatccattcgatcccgcggcgtataagtactggggccaagggacccaggtcaccgtctcctcagcggccgcatacccgtacgacgttccggactacggttcccaccaccatcaccatcactag

SEQ ID NO: 2 - DNA, 423 bp, Artificial Sequence
  It is synthesized.
gtgcagctgcaggagtctggaggaggctcggtgcaggctggagggtctctgacactctcctgtgtatgctctggatacatacccagtaaggcctggttccgccaggctccagggaaggagcgcgaggcggtcgcagcgattgatgggcctggtaggataaggtacgtagactccgtgaacggccgattcaccatctcccaagacattgtcaagaagactctcgttctcgaaatgaacagcctgaaacctgaggacactgccatgtactactgtgcggcagatcccaggttgtctactaataactggtcgcgagcccccgagtatcgctactggggccaggggacccaggtcaccgtctcctcagcggccgcatacccgtacgacgttccggactacggttcccaccaccatcaccatcactag

SEQ ID NO: 3 - DNA, 414 bp, Artificial Sequence
  It is synthesized.
gagtctgggggaggcttggtgcagcctggggggtctctgagactctcctgtgcagcctctggattcaccttcagtaactatgccatggaatgggttcgccaggctccagggaagggactcgagtgggtctcaggcattaatagtggaggtattggtacatactatgcagactccgtgaagggccgattcaccatctccagagacaacgccaagaacacgctgtatctgcagctgaacagcctgaaaactgaggacacggccatgtattactgtgcaaaggggtacggtaatatggtggtggctggtatcacaagtaactaccgaggccaggggacccaggtcaccgtctcctcagcggccgcatacccgtacgacgttccggactacggttcccaccaccatcaccatcactag

SEQ ID NO: 4 - DNA, 408 bp, Artificial Sequence
  It is synthesized.
gtgcagctgcaggagtctgggggaggcttggtgcagcctggggggtctctgagactctcctgtgcagcctctggattcaccgtcagtagaactgccatgagctgggtccgccaggctccagggaagggactcgagtgggtcgccactattggtactagtgatagtatcacagtctatgcagactccgtgaagggccgattcaccatctccagagacaacgccaagaacacgctgtatctgcaattgaacagcctgaaaactgacgacacggccgtgtataattgtgcaaaatgtttccgttactgcccaccaccccttaccccggggacccaggtcaccgtctcctcagcggccgcatacccgtacgacgttccggactacggttcccaccaccatcaccatcactag

SEQ ID NO: 5 - DNA, 426 bp, Artificial Sequence
  It is synthesized.
gagtctggaggaggctcggcgcaggctggagggtctctgagactctcctgtccagcctctggattgacttccagtaccgcctgcttggcctggtttcgccaggctccagggaaggagcgcgagggggtcgcacgtatacgttctctttttggtagcacatggtatgccgactccgtgaagggccgattcaccatctcccaagacaccaccaagaatatgctgtatttacaaatggacagcctgaaacctgaggacactgccatgtactactgtgcggcagggcggacgggagtctgctattcaggctcttggtcacctgatgagtttaactactggggccaggggacccaggtcaccgtctcctcagcggccgcatacccgtacgacgttccggactacggttcccaccaccatcaccatcactag

SEQ ID NO: 6 - DNA, 420 bp, Artificial Sequence
  It is synthesized.
gagtctggaggaggctcggtgcaggctggagggtctctgagactctcctgtgttgcctctggattcatctacacctacagtagttactgcatgggctggttccgccaggctccagggaaggagcgcgagggggtcgcaactattgatagagatcgtagcacaacctacgcagactccgtgaagggccgattcaccatctcccaagacaacgccaagaacactctgtatctgcaaatgaacagcctgaaacctgaggacactgccatgtactactgtgcggcagtccgtagtagtggttactgctacatcgcccagtggtataagtactggggccaggggacccaggtcaccgtctcctcagcggccgcatacccgtacgacgttccggactacggttcccaccaccatcaccatcactag

SEQ ID NO: 7 - DNA, 423 bp, Artificial Sequence
  It is synthesized.
gagtctgggggaggctcggtgcagactggagggtctctgagactctcctgtatagtatctggatacacctacagtcgcaagaccgtgggctggttccgccaggctccagggaaggagcgcgagggggtcgcagcggtttattctgcaggtggtactagtacgacatactataccgactccgtgaagggccgattcaccatctccctagacaccaccaagaacacgatttatctacaaatgaacagcctgagatatgaggacactgccatgtactattgtgcgacaggtttcggcggtgctaactattacgacaaagcacggtataagtactggggccaggggacccaggtcaccgtctcctcagcggccgcatacccgtacgacgttccggactacggttcccaccaccatcaccatcactag

SEQ ID NO: 8 - DNA, 423 bp, Artificial Sequence
  It is synthesized.
gagtctggaggaggctcggtgcaggctggagggtctctgagactctcctgtgcagcctctggatacacctacagtagcacctacatggcctggttccgccaggctccagggaaggagcgcgagggggtcgcaagtatttatactggtgatggttggacatactatgccaactccgtgaagggccgattcaccatctcccgagacaacgccaagaacacggtgtatctgcaaatgaacagcctgaaacctgaggacactgccatgtactactgtgcggccaaaacccgtgcagtcggtgggacctggtcatccccaaacatatatacgtactggggccaggggacccaggtcaccgtctcctcagcggccgcatacccgtacgacgttccggactacggttcccaccaccatcaccatcactag

SEQ ID NO: 9 - DNA, 420 bp, Artificial Sequence
  It is synthesized.
gtgcagctgcaggagtctggaggaggctcggtgcaggctggagggtctctgagactctcctgtgtagcctctagatccagcgacaactacatgggatggttccgccaggctccagggaaggagcgcgagggggtcgccgccatttggggtggcaccgcatactatgccgactccgtcgagggccgattcaccatctcccaagacaacgccaagaacacagtgtatctgcaaatgaacagcctgaaacctgaggacactgccatgtactactgtgcgacaggtttcttgaaaggtggtagttggtcgagggcagagaggtttcattactggggccaggggacccaggtcaccgtctcctcagcggccgcatacccgtacgacgttccggactacggttcccaccaccatcaccatcactag

SEQ ID NO: 10 - DNA, 423 bp, Artificial Sequence
  It is synthesized.
gtgcagctgcaggagtctgggggaggctcggttcaggctggagggtctctgagactctcctgtgcagcctctggacagaccgccttcatggcctggttccgccaggctccagggcaggagcgcgagggggtcgcagctattgatactcgtgctttaagcacatactatggcgactccgtgaagggtcgcttcaccatctccaaagacattttcaagaacactctgtatctgcaaatgaacagcctgaaacctgaggagactgccatgtactactgtgcagcgagaaaaaaaccggggagcagcctcttgcaaccgcaatcgtatgtttactggggccaggggacccaggtcaccgtctcctcagcggccgcatacccgtacgacgttccggactacggttcccaccaccatcaccatcactag

SEQ ID NO: 11 - DNA, 435 bp, Artificial Sequence
  It is synthesized.
gtgcagctgcaggagtctgggggaggctcggtgcaggctggagggtctctgagactctcctgtgcagcctctacatacgcctacagtagcacctacatggcctggttccgccaggctccagggaaggagcgcgagggggtcgcaagtatttatactggtgatggttggacatactatgccaactccgtgaagggccgattcaccatctcccgagacaacgccaagaacacggtgtatctgcaaatgaacagcctgaaacctgaggacactgccatgtactactgtgcggccaaaacccgtgcagtcggtgggacctggtcatccccaaacatatatacgtactggggccaggggacccaggtcaccgtctcctcagcggccgcatacccgtacgacgttccggactacggttcccaccaccatcaccatcactag

SEQ ID NO: 12 - DNA, 432 bp, Artificial Sequence
  It is synthesized.
gtgcagctgcaggagtctggaggaggctcggtgcaggctggagggtctctgagactctcctgtgcagcctctgtatacagacggcataactactgcatgggctggttccgccagcgtccagggaaggagcgcgaggagatcgcaatttttgataggggtgatagcacaagctacgcagactccgtgaagggccgattcaccatctcccaagacaacgccaagaatatgttgtatctgcaaatgaacagcctgaaacctgaggacactgccatgtactactgtgcgacatcatacccaagggtggtaattggtacctgtacaggtggggactttcgtgtctggggccaggggacccaggtcaccgtctcctcagcggccgcatacccgtacgacgttccggactacggttcccaccaccatcaccatcactag

SEQ ID NO: 13 - DNA, 441 bp, Artificial Sequence
  It is synthesized.
gtgcagctgcaggagtctgggggaggctcggtgcaggctggagggtctctgagactctcttgtgcagcctctggcgctccctacagtacctattctatgggctggatccgccaggctccagggaagcagcgcgaggcggtcgcagctattgatagtgatgggaacataagctacgcagactccgtgaagggccgattcaccatctcccaagacaacctcaagaacactctgtatctacaattgaacaaactgaaacctgaggacactgccatgtactactgtgcggcagattatccagcaacctcagggggtagtggtggtttcttagccgcactgcttcataagtactggggccaggggacccaggtcaccgtctcctcagcggccgcatacccgtacgacgttccggactacggttcccaccaccatcaccatcactag

SEQ ID NO: 14 - DNA, 441 bp, Artificial Sequence
  It is synthesized.
gtgcagctgcaggagtctggaggaggctcggtgcaggctggagggtctctgagactctcctgtgcaacctctggatttcccttcagtacttactgtatggggtggttccgccaggttccagggaaggagcgcgagggggtcgccggtatttatattagtcatggtcagacatattatgccgactccgtgaagggccgattcaccatctcccgagacgacaagaagaagacgctgtatctacaaatggaagccctgaaccctgaggacactgccatgtattactgtgcggcaggagaccggtggtactgccccgcggactcatggtcccggccagaaaggtatccgttctggggccaggggacccaggtcaccgtctcctcagcggccgcatacccgtacgacgttccggactacggttcccaccaccatcaccatcactag

SEQ ID NO: 15 - DNA, 420 bp, Artificial Sequence
  It is synthesized.
gtgcagctgcaggagtctggaggaggctcggtgcaggctggggggtctctgagactctcctgtgcagcctctggattcacattcagtatgaacggcatgagctgggtccgccaggctccagggaaggggctcgagtgggtctcagcgattactactggtggtagtgtggcaacgtatgcggactccgtgaagggccggttcaccatctccagagacaatgccaagaacacgctgtatctccaaatgaacaacctgaaaactgaagacactgccgtgtattactgcgccacagggaatcaacgtaggccggcacagcacgtgggtgtgtggggccaggggacccaggtcaccgtctcctcagcggccgcatacccgtacgacgttccggactacggttcccaccaccatcaccatcactag

SEQ ID NO: 16 - DNA, 420 bp, Artificial Sequence
  It is synthesized.
gtgcagctgcaggagtctggaggaggctcggtgcaggctggagggtctctgagactctcctgtacagcctctggatacaccagcagtatgggctggttccgccaggctccagggaaggagcgcgagggggtcgcagctatcggcattgttaacgcagtctacgcggattccgtgaagggccgattcaccatctccaaagacaacgccgagtacactctctatctgcaaatgaacagcctgaaacctgaggacactgccatgtactactgtgcggcagtacggcgcctacaaaaaaaaaaccaactggcttcggatgagtatatgtactggggccaggggacccaggtcaccgtctcctcagcggccgcatacccgtacgacgttccggactacggttcccaccaccatcaccatcactag

(19) United States
(12) Patent Application Publication
(10) Pub. No.: US 2020/0024365 A1
(43) Pub. Date: Jan. 23, 2020

(54) mRNA-ENCODED NANOBODY AND APPLICATION THEREOF

(71) Applicant: SOUTHEAST UNIVERSITY, Nanjing (CN)

(72) Inventors: Wei XIE, Nanjing (CN); Rui HAO, Nanjing (CN); Xuechen ZHOU, Nanjing (CN); Yunyan ZHOU, Nanjing (CN); Zhipeng SU, Nanjing (CN)

(21) Appl. No.: 16/484,977

(22) PCT Filed: Apr. 27, 2017
(86) PCT No.: PCT/CN2017/082096

§ 371 Date: Aug. 9, 2019

(30) Foreign Application Priority Data
Apr. 7, 2017 (CN) 201710224513.4

(51) Int. Cl. C07K 16/40; C12N 15/88; C12N 15/85

(52) U.S. Cl. CPC C07K 16/40; C12N 15/88; C12N 2800/107; C07K 2317/569; C07K 2317/22; C12N 15/85

(57) ABSTRACT

It relates to a nanobody encoded by a mRNA and an application thereof, and more particularly, to the field of nanobody drugs. In order to solve the problem above, the invention provides a novel concept and a method system for regulating an intracellular protein using an mRNA-encoding nanobody. The invention discloses a nanobody encoded by a mRNA, and encoding information carried by the mRNA is recognized, translated and expressed to a single-chain nanobody that can be bound to a targeting protein in a cell. By adopting the method, a function of a disease-related protein in a cell can be effectively and specifically interfered with, thus achieving the object of disease treatment.

### TECHNICAL FIELD

The present invention relates to a nanobody encoded by a mRNA and an application thereof, and belongs to the field of biomedicine, and more particularly, to the field of nanobody drugs.

### BACKGROUND

With the progress of biomedical research, a large number of protein dysfunctions related to disease occurrence have been discovered, of which most (more than 90%) pathogenic proteins are located in cells. However, since it is difficult for an ordinary monoclonal antibody to enter in a cell effectively, the existing antibody drugs all realize their therapeutic effects by being bound to a targeting protein located on a cell surface or outside a cell. A number of extracellular targeting proteins is very limited, and due to this limitation, the targeting proteins recognized by the existing antibody drugs in the market are highly concentrated. On the one hand, an indicational range of the antibody drugs is greatly limited, and many diseases lack suitable antibody drugs; and on the other hand, limited targets also lead to high homogenization of research and developmental works of pharmaceutical companies, resulting in excessive competition in the industry and waste of social resources. A method capable of conveniently, efficiently, safely, specifically and bidirectionally (comprising activation and inhibition) regulating an activity of an intracellularly functional protein will open a new stage in the development of biopharmaceutical industry.

Expression of a functional protein in a cell using a plasmid-represented DNA or a virus as a vector is a common method of gene therapy, and although this method has a high transfectional and expressional efficiency, a risk of other diseases caused by recombination of a DNA and a targeting cell genome cannot be avoided. For example, an exogenous DNA may be inserted in a normal gene, resulting in mutations or even complete destruction of the expression of the gene, and if the inserted gene is a relatively important functional gene, this change will cause significant damage to cell function and even lead to cell transformation to develop cancers. Related carcinogenic risks cannot be avoided theoretically using a DNA-based plasmid or virus as a tool, which is also an important reason why the gene therapy has not been fully promoted. In contrast, a RNA cannot be reversely transcribed into a DNA after entering a cell. Therefore, a stability of a cell genome cannot be damaged theoretically. It can be said that a RNA therapy fundamentally avoids the carcinogenic risk of the gene therapy and is more suitable for the safety requirements of clinical medication. In addition, the DNA needs to enter into a cell nucleus to be transcribed and expressed, which is difficult to be transfected, and to some extent, the cell needs to be in an active division cycle, so that the DNA can enter the cell nucleus. However, the RNA can be expressed as long as entering a cytoplasm without entering the cell nucleus for transcription and without depending on the cell cycle. Under the same transfection condition, the RNA is more efficient than the DNA in intracellular expression and has less difficulty in clinical application.

### SUMMARY

In order to solve the problem above, the present invention provides a novel concept and a method system for regulating an intracellular protein by a nanobody encoded by a mRNA.

In the 1990s, people found an antibody with natural deletion of a light chain in bodies of cartilaginous fishes and Camelidae animals, and found that such antibody often existed in a form of heavy chain dimer. Such antibody is called a heavy chain antibody. Through further research, it is found that in a camel-derived heavy chain antibody, a variable domain of heavy chain antibody bound to an antigen can be separately and stably expressed, and has a good specificity and affinity for antigen binding, and the variable domain of heavy chain antibody (V_{H}H) is called a single-domain antibody (sdAb) or a nanobody. The nanobody is the smallest antigen binding protein that has been known and can be stably expressed, with a molecular size being only one tenth of that of a conventional IgG antibody, and has the advantages of stable structure and strong specificity.

On this basis, we firstly disclose a nanobody encoded by a mRNA in the present invention, and encoding information carried by the mRNA is recognized and translated into a cell to express a single-chain nanobody that can be bound to a targeting protein. That is to say, a method and a tool system for expressing a mRNA encoding a specific nanobody in a targeting cell are described in the present invention. This tool system comprises: at least a mRNA molecule that is transcribed in vitro and encodes the nanobody, a transfection tool for assisting the mRNA molecule to enter a cell, and an auxiliary agent for enhancing a transfection complex to recognize a specific cell and improving a transfection efficiency thereof. After the mRNA molecule is transported in the targeting cell, the mRNA molecule can be recognized by a translation machine in the cell, and a single-chain nanobody bound to a specific targeting protein is expressed. According to different binding modes of the nanobody and the targeting protein, conformation of the targeting protein is changed in different forms, and thus realizing the effect of inhibiting or activating a biological function of the targeting protein.

For the construction of the mRNA molecule to encode the nanobody recognizing the specific targeting protein disclosed in the present invention, a stability and a translational efficiency of the mRNA in vivo are improved by introducing certain chemical modification and nucleotide sequence, the mRNA is transferred into the specific cell, and a method and a tool system of intracellular expression of a corresponding nanobody are realized. The method and the tool system are particularly suitable for medical clinical treatment of diseases caused by dysfunction of a specific intracellular protein. The tool system described by the present invention is used for treating a cell with a specific protein of functional defect, and according to different results caused by the binding of the generated nanobody and the targeting protein, an indication treated comprises two aspects: the first aspect is that a nanobody encoded by a mRNA inhibits a function of the targeting protein through binding, and is used for treating diseases caused by high expression or over activation of a pathogenic gene in a cell; and the second aspect is that the nanobody encoded by a mRNA stabilizes the conformation of the targeting protein in a highly active state, thus enhancing the function of the protein, which is used for treating the functional defect caused by insufficient expression or decreased activity of an intracellular targeting protein.

The nanobody mentioned in the present invention refers to a variable structure domain of the camel-derived heavy chain antibody, which can be specifically and closely bound to an antigen epitope on the targeting protein. Due to a single chain protein and a relatively stable structure, common nanobodies are all obtained by high expression of microorganisms such as Colibacillus or yeast. In order to obtain the nanobody regulating the function of the targeting protein, a purified antigen protein or an antigen epitope structure domain of the protein is usually mixed with a suitable immune adjuvant, and then camel animals such as a two-humped camel, an alpaca, etc. are injected for multiple times, and a stimulated immune system of the animal generates a B cell capable of secreting a specific heavy chain antibody of the antigen protein through processes of cell activation, differentiation and maturation; total B cells in peripheral blood of an immune animal are collected, total RNAs are extracted and a cDNA library is reversely transcribed, or the B cell capable of being bound to a target antigen is screened by a flow cytometry, and total RNAs are extracted and a cDNA library is reversely transcribed; the cDNA library is used as a template, encoding genes of a single-domain variable domain of heavy chain antibody (V_{H}H) is amplified by nested PCR with a specific primer (sequence 1), and a nanobody library capable of recognizing a targeting antigen protein is screened by a phage display technology; these genes are transferred into the colibacillus to establish a nanobody strain that can be efficiently expressed in the colibacillus, an antibody strain with higher affinity to the antigen protein is determined by an ELISA method, and the encoding genes of these nanobodies are sequenced; gene sequences of cloned strains are analyzed according to sequence alignment software, redundant clones of FR1, FR2, FR3, FR4, CDR1, CDR2 and CDR3 with the same sequences are removed, similar sequences of the CDR3 region (sequence similarity >80%) are classified into a group, the nanobody with higher affinity in each group is selected to be cloned, and a corresponding nanobody is obtained through high expression and purification in the Colibacillus; and a biochemical method is used to examine effects of different nanobodies on the function of the antigen protein, so as to obtain the nanobody capable of activating or inhibiting the function of the targeting protein.

A gene sequence encoding the functional nanobody is obtained through sequencing, and the data can be used as basic sequence information for synthesizing the mRNA encoding the nanobody by a biological mean in the present invention. The biological mean usually refers to a method for obtaining the mRNA by in vitro transcription, and the method refers to a process of generating the mRNA by imitating an in vivo transcription process with a DNA as a template and a NTP as a raw material relying on a RNA polymerase in an in vitro cell-free system.

A method for obtaining a gene sequence of the specific nanobody and the corresponding mRNA is described in the method, the preferred solution is to clone a DNA of the single-domain variable domain of heavy chain antibody capable of being specifically bound to the targeting protein, which is screened from a camel-derived immune single-domain heavy chain antibody library by the flow cytometry and phage display technologies, or a high-throughput sequencing technology (or Next Generation Sequencing, NGS) combined with a bioinformatics analysis method, sequencing, expression and activity recognition are performed to obtain the functional nanobody capable of being specifically bound to the targeting protein and the gene sequence thereof, and the mRNA encoding the nanobody is obtained by chemical synthesis or in vitro transcription according to the obtained gene sequence.

Generally speaking, the gene sequence of the nanobody obtained by the phage display technology is usually located on a phage display plasmid, comprising pHEN1, pHEN4, pMES4 or pMESy4, due to a low copy number of these plasmids, an optimizational method is to transfer a gene encoding nanobody on other plasmids more suitable for in vitro transcription by subcloning, comprising high copy plasmids such as pUC18 and pUC19, series plasmids of pT7Ts and pGEM-1, or pSP64 plasmids; and a large number of DNA templates for in vitro transcription can also be obtained by a PCR method.

Further, the present invention also discloses that a mRNA molecule carries at least one modification to enhance a stability of the mRNA molecule. The preferred solution is that the mRNA molecule carries various modifications to enhance a stability of the mRNA molecule. In some situations, the modification enhancing the stability comprises: replacing a natural unmodified nucleotide by a chemically modified nucleotide, increasing a content of GC bases in the mRNA without affecting composition of an amino acid encoding the nanobody, and introducing a non-encoding sequence (UTR) at two ends of the mRNA to improve a stability of the mRNA. The increase of the content of the GC bases in the mRNA mentioned here refers to that a proportion of the GC bases shall be increased as much as possible compared with an AU base pair.

For example, in order to improve a stability of a mRNA transcription product, the present invention recommends that specific non-encoding sequences, i.e., 5′-UTR and 3′-UTR, are added to an upstream and a downstream of an encoding sequence without affecting an amino acid sequence of an encoding protein. One of related situations is to add a long poly-A tail at a 3-terminal of the mRNA, thus improving the stability of the mRNA. Under certain circumstances, a length of the poly-A tail is not less than 100, 200, 300 or 400 nucleotides. A method for adding the long ploy-A tail comprises directly adding a poly-A at a terminal of the mRNA transcription product by a poly-A polymerase, directly introducing the poly-A at a 3′-terminal of the DNA template transcribed in vitro, or directly connecting the poly-A with the 3′-terminal of the mRNA transcription product by a mRNA ligase. In particular, since the length of the poly-A tail can regulate a half-life of the mRNA transcription product in vivo, and in some situations, a time for the mRNA to express the nanobody in a cell can be controlled by regulating the length of the poly-A tail carried by the mRNA transcription product. Except for the poly-A tail, another common situation is to introduce specific 5′-UTR and 3′-UTR regions to an upstream and a downstream of the gene encoding the nanobody, especially more C bases are repeated at a downstream of a mRNA termination codon, which has been proved to improve a stability of the mRNA. The present invention recommends a non-encoding region of the mRNA derived from other natural proteins with higher stability, such as the 5′-UTR and 3′-UTR regions of the mRNA of a protein such as collagen, globin, actin, tubulin, GAPDH, histone, etc., and the introduction of these non-encoding regions at the upstream and the downstream of the encoding sequence of the nanobody can also improve the stability of the corresponding mRNA transcription product. According to our research, a homology sequence is extracted from an UTR region at the upstream and the downstream of the encoding region of the mRNA of various high-stability proteins, the following two new sequences (the sequences are as follows) obtained through screening can significantly improve the stability of the mRNA, and under the same conditions, the half-life of the mRNA in a cell is more than twice by the addition of the non-encoding sequence.

Added sequence of 5′UTR:
5′ACCGCCGAGCCGTTTCCGGGACCCGTGCTTCTGACCCTACCGCCTTCG
CCAGCATCCTCAAACCGCCACC 3′
Added sequence of 3′UTR:
GGTGGCTCCTGCCACTCTGCCCCTTGCCCTCCCCTGCCCCCTTTCTTGCT
GTCCAACTTACCTGAAAGGTTTGAAGGCTCCCTGAGTCCCTTTACTTGAC
TGGGGGATATTAGGAAGGGCCTTTACCATGTGGAACCTGCTTAATAAAAA
ACATTTATTTTTCATAGC

It shall be noted that the four methods for optimizing an encoding sequence of the mRNA molecule above can be used separately or in combination, as well as an object of making the generated mRNA with higher stability and translational efficiency.

In most situations, the mRNA in the tool system of the present invention carries one or more modifications to enhance the stability of the mRNA for prolonging a half-life of the mRNA molecule, thus improving an efficiency of the mRNA molecule to be translated and expressed in targeting cells to generate the nanobody. The chemical modification of the mRNA molecule here refers to the modification of components forming the mRNA—nucleotides, i.e., nucleotides without modified groups under natural conditions are replaced with nucleotides with modified groups. Specifically, the nucleotide is composed of three parts: nitrogenous base, ribose and phosphoric acid, in order not to affect the generation of the mRNA during in vitro transcription, the chemical modification here is mainly located in a base part thereof. Commonly used modified bases are comprised but not limited to analogues or derivatives of purine (adenine A and guanine G) and pyrimidine (cytosine C, uracil U, thymine T) with modified bases such as methylation, acetylation, hydrogenation, fluorination and vulcanization. The most commonly used modified bases comprise, but are not limited to, for example, pseudouridine (ψ), N6-methyladenine (N6mA), inosine (I), methyluridine (mU), 5-methylcytosine (5mC), 5-hydroxymethylcytosine (om5C), dihydrouridine (DHU, D), ribothymidine (rT), 7-methylguanosine (m7G), etc.

In particular, since numbers of U and C bases are inversely proportional to the stability of the mRNA, one of situations recommended by the present invention is that uracil is replaced by as much the pseudouridine (ψ) as possible, and cytosine is replaced by as much the methylcytosine as possible, and ratios of U and C replaced by the pseudouridine and the methylcytosine in a mRNA transcription product exceed 20%, 30% and 40%, and even 100% under an ideal condition.

The present invention recommends that the base substitution for enhancing the stability of the mRNA is performed in the encoding sequence by means of molecular biology without affecting the amino acid sequence of the encoded protein. The stability of the mRNA molecule is decreased with the increase of numbers of cytidines (C) and uridines (U) contained, while the stability of the mRNA without C and U bases is relatively high. It shows that a content of A shall be increased as much as possible in an AU base pair, while a content of G shall be increased as much as possible in a CG base pair. Under the situation recommended by the patent, contents of C and U bases in the mRNA sequence can be reduced without affecting the amino acid sequence of the protein. A specific situation is that the contents of C and U in the whole mRNA sequence can be reduced by replacing an original codon with more C and U with a codon with the same amino acid but less C and U, thus making the mRNA molecule more stable. For example, a codon GGU or GGC of glycine (Gly) can be adjusted to GGA or GGG, a codon GCU or GCA of alanine (Ala) can be adjusted to GCA or GCG, a codon GUU or GUC of valine (Val) can be adjusted to GUA or GUG, a codon CUU or CUC of leucine (Leu) can be adjusted to UUG or CUG, a codon AUU or AUC of isoleucine (Iso) can be adjusted to AUA, a codon UCU or UCC of serine can be adjusted to UCA or UCG, a codon CCU or CCC of proline (Pro) can be adjusted to CCA or CCG, a codon ACU or ACC of threonine (Thr) can be adjusted to ACA or ACG, a codon CGU or CGC of arginine (Arg) can be adjusted to CGA or CGG, etc. and these substitutions can reduce the contents of C and U in the encoding region to improve the stability of the mRNA.

Meanwhile, the present invention further preferably discloses that the mRNA molecule carries at least one modification to enhance a translational efficiency of the mRNA molecule. The preferred solution is that the mRNA molecule carries various modifications to enhance the translational efficiency thereof. In some situations, the modification to enhance the translational efficiency comprises: introducing a ribosome-recruiting cap (5′ cap) structure at a 5′-terminal of the mRNA, introducing a Kozak sequence and other sequences enhancing the translational efficiency to an upstream of the encoding region of the nanobody, and using an amino acid codon with higher translational efficiency without affecting the amino acid composition of the encoded nanobody.

Considering that the codon used in the encoding region of the nanobody is corresponding to a used frequency of a tRNA, in order to improve the translational efficiency of the mRNA transcription product, the method of the present invention recommends that a tRNA codon with higher use frequency in human cells is used to replace those codons with lower use frequency. For example, the CUG with the highest used frequency is selected from six codons encoding leucine (Leu), and the GUG with the highest used frequency is selected from four codons encoding valine (Val); and the CAG with the highest used frequency is selected from two codons encoding glutamine, etc.

In addition, in order to promote the recognition of the ribosome to the mRNA transcription product and improve the translational efficiency, the 5-terminal of the mRNA molecule needs to have a specific 5′-cap structure. 5′-terminal capping can not only improve the stability of the transcription product, but also can improve the recognition efficiency of a translation machine, thus increasing an expression level of the nanobody encoded by a mRNA in a cell. Commonly used cap-structure modified bases comprise but are not limited to 7-methylguanine (m7G(5′)ppp(5′)G, 7-methylguanylate), Thermo Fisher's ARCA-cap (Anti-Reverse Cap Analog), etc., and the latter can increase a probability of mRNA synthesis in a right direction. Capping of 5′-terminal of the mRNA can be realized by replacing partial normal GTP with such modified guanine in a response system transcribed in vitro, and a working concentration of the modified guanine is recommended to be 1 mM to 4 mM, and the dosage is 2 times to 8 times of the concentration of the normal GTP.

The method for introducing base modification in the present invention is that the nucleotides with modified groups are additionally added to the transcriptional system, or naturally unmodified nucleotides are completely replaced with the nucleotides with modified groups, so that these modified nucleotides participate in the synthesis of the mRNA transcription product under the action of the RNA polymerase. It shall be noted that the base modification above can be used separately or in combination with many methods, with the object of making the generated mRNA have higher stability and translational efficiency.

In some cases, in order to further improve the stability of the mRNA transcription product of the encoded nanobody in the present invention, special substances can be added to the tool system to prolong the half-life of the mRNA in a cell. Such reagents can be special proteins or nucleic acid fragments, such as a poly-A binding protein used to protect the 3′-terminal sequence of the mRNA molecule, or a DNA or mRNA fragment complemented with a certain sequence of the mRNA transcription molecule. Before the mRNA is transfected into a cell, such substances are mixed with the mRNA, the substances can be directly or indirectly interacted with the transcribed mRNA, and after the mixture is transferred into the cell, chances of degrading the mRNA bound to a protective molecule by the nuclease are reduced, thus the mRNA can remain in the cell for a longer time. It shall be noted that different protective molecules can be used separately or in combination with many methods, with the object of making the generated mRNA have higher stability and longer half-life in a cell.

Further, the present invention further preferably discloses that the mRNA molecule carries at least one modification to reduce an immunogenicity of the mRNA molecule. The preferred solution is that a possibility of causing a body immune response is reduced by various modifications. In some situations, modification to reduce the immunogenicity of the mRNA comprises: removing a phosphorylation group, especially a triphosphate group, causing the immunogenicity at the 5′-terminal of the mRNA molecule transcribed or chemically synthesized in vitro by phosphatase treatment; and preventing the mRNA molecule from forming a secondary structure of a double-stranded mRNA type with strong immunogenicity, etc. by introducing N6-methyladenosine, 5-methylcytidine and other 2-O-methylated nucleotides into the mRNA molecule.

Cells have an innate immune system, and in the case of infection by pathogenic molecules, invading substances can be degraded by activating a series of complex inflammatory responses. Many mammalian cells express a variety of pattern recognition receptors (PRRs), which can recognize, bind and degrade exogenous mRNA entering a cell, which is an important reason for a low translational efficiency of the mRNA transcribed in vitro in a cell. In most situations, the mRNA in the tool system of the present invention carries one or more modifications to reduce the immunogenicity of the mRNA, thus improving the efficiency of the mRNA molecule to be translated and expressed in targeting cells to generate the nanobody.

It has been reported that phosphorylation modification at the 5′-terminal of the exogenous mRNA molecule can affect the immunogenicity of the mRNA to an innate immune system, and the mRNA with 5′-triphosphorylational modification is easier to be degraded than the mRNA without the phosphorylational modification. According to this characteristic, it is recommended by the present invention that phosphatase treatment can be used to treat a phosphorylation group, especially a triphosphate group, with the immunogenicity caused by removing the 5′-terminal of the mRNA molecule transcribed or chemically synthesized in vitro to reduce the immunogenicity. In addition, natural mRNA in an eukaryotic cell often carries some modified nucleotides, such as N6-methyl adenosine, 5-methyl cytidine, and other 2-O-methylated nucleotides, and although the number is small, the modified nucleotides can prevent the mRNA molecule from activating an innate immune response. Accordingly, the present invention recommends that the modified nucleotides comprising N6-methyladenosine, 5-methylcytidine and 2′-O-methylated nucleotides are introduced into the mRNA molecule transcribed in vitro, and these modified bases can prevent the mRNA molecule from forming the secondary structure of the double-stranded mRNA type with strong immunogenicity, thus reducing degradation of the mRNA molecule by the innate immune response and prolonging the half-life in a cell. It shall be noted that different modification methods can be used separately or in combination with many methods, with the object of making the generated mRNA have lower immunogenicity and longer half-life in a cell.

Meanwhile, the present invention further preferably discloses that an upstream and a downstream of a mRNA-encoding protein are modified with intracellularly localizational signals. That is to say, a nanobody encoded by a mRNA molecule can be located in a specific cell region or organelle according to cell distribution of the targeting protein, for example, in some situations, the generated nanobody can be located in a specific subcellular structure such as a cytoplasm, a cell nucleus, a mitochondria, an endoplasmic reticulum, or a Golgi apparatus as required.

The encoding region of the mRNA molecule transcribed in vitro used in the present invention can contain a specific cell positioning signal, and according to different cell regions where the binding targeting protein is located, the nanobody generated by translation of the mRNA molecule can be located in the corresponding specific cell region to increase a recognition effect of the nanobody on the targeting protein. The localizational signal here refers to a special amino acid sequence obtained by encoding and translation of the mRNA, such as a signal peptide, a leader peptide, a classificational signal, a localizational signal, etc., which can be located at an N-terminal or a C-terminal of the nanobody according to requirements, comprising but being not limited to: a nuclear localizational signal (NLS, such as PKK KRKV, PQKKIKS, QPKKKP, RKKR, etc.) for locating the nanobody in the cell nucleus; a nuclear export signal (NES, such as LxxxLxxLxL) for locating the nanobody in the cytoplasm; an ER-retention signal (such as KDEL,DDEL,DEEL,QEDL, RDEL and other sequences) for locating the nanobody on the endoplasmic reticulum; an endosome localizational signal (such as sequence MDDQRDLISNNELP) for locating the nanobody to an endosome; a mitochondrial targeting signal for locating the nanobody in the mitochondria; a peroxisomal targeting signal (PTS, such as SKL) for locating the nanobody to a peroxisome, etc.

As another disclosure, we further disclose an application of the nanobody encoded by a mRNA above in preparing a regulation of a function of an intracellular targeting protein in the present invention.

Further, we disclose that the mRNA encoding the nanobody enters a cell through a transporter.

The tool system of the present invention also comprises the transporter carrying a mRNA transcript to enter a targeting cell. The targeting cell refers to a cell with a functional defect of the targeting protein, expression of the nanobody encoded by a mRNA in these cells can regulate the activity of the targeting protein and restore the normal function of a cell. The transporter here comprises various forms of drug carriers that help nucleic acid substances to be ingested, and the composition of the carrier can deliver the mRNA molecules with different sizes into the targeting cells. In the transporter, the mRNA molecule can be bound to one or more chemical reagents, so as to be packaged into a form capable of entering the targeting cell. In selection of a suitable chemical reagent, not only biological and chemical properties of the mRNA transported shall be considered, but also the use condition in application of a therapeutic solution and a biological environment and other factors exposed by the mRNA molecule after application shall be considered. The transporter does not destroy the biological activity of the mRNA while wrapping the mRNA during application. In some situations, the transporter has a certain tendency to the targeting cell, the ability to bind to the targeting cell is stronger than that of other non-targeting cells, and under ideal circumstances, the transporter used in the present invention can specifically transfer the mRNA into the targeting cell without affecting other normal cells without regulation.

According to requirements, the transporter can be a liposome vector, or other reagents facilitating the nucleic acid to enter the targeting cell. The suitable transporters comprise but are not limited to: liposomes; nanoliposomes with ceramide; liposomes with special lipoproteins, such as liposomes with a ligandin apolipoprotein-B or apolipoprotein-E, which can be bound to the targeting cell expressing a low density lipoprotein receptor to increase mRNA-carrying transporters entering the corresponding targeting cells; nanoliposomes; nanoparticulates comprising calcium phosphate nanoparticulates, silica nanoparticulates, nanocrystalline particulates, biological nanoparticulates, semiconductor nanoparticulates, etc.; polyarginines; starch transport systems; micelles; emulsion; capsid proteins of certain viruses, etc., and certain poly compounds can also be used as the RNA transporters. A transporter system can be used to preferentially target the mRNA encoding the nanobody to a variety of targeting cells, and suitable targeting cells comprise but are not limited to: liver cells, epithelial cells, endothelial cells, lung cells, bone cells, stem cells, mesenchymal cells, nerve cells, myocardial cells, adipocytes, immune cells, ovarian cells, testicular cells, tumor cells, etc.

In some situations, in order to facilitate observation, the transporter of the present invention also comprises a biological indicator easy to be detected, so as to show that the mRNA encoding the nanobody successfully enters the targeting cell. The commonly used detection indicator comprises isotope labeling, fluorescence labeling or other materials commonly used in the in vivo and in vitro experiments. The detection substance can be covalently connected with the mRNA molecule for tracing the mRNA molecule in a cell or a tissue; and can also be encoded by the mRNA sequence (such as a fluorescent protein, a luciferase, etc.) to show the translation of the mRNA molecule in a cell.

Further, we preferably disclose that the transporter is added with a ligand bound to a surface marker of a targeting cell to be regulated. That is to say, the ligand bound to a surface marker protein of the targeting cell can be added to the transporter to increase a targeting ability of a transport complex to the targeting cell, the mRNA can be transferred into the specific cell of the specific tissue, and the nanobody of a specific functional protein can be translated and expressed in a cell. In some situations, a vector carrying the mRNA encoding the nanobody can enter a tissue and a cell through a passive method of natural diffusion, while in other situations, the vector carrying the mRNA has additional components that can be recognized by specific target cell, thus enabling the vector to selectively enter a specific cell. The preferred solution is that the vector carrying the mRNA in the present invention can have the ligand capable of increasing the binding of the complex to one or more targeting cells, in some situations, the ligand for localization is the apolipoprotein-B or the apolipoprotein-E, and these ligands can be bound to the targeting cells expressing the low density lipoprotein receptor; and in other cases, the localization ligand is an antibody capable of being bound to the surface marker protein of the targeting cell. These ligands can be bound to the surface marker protein of the targeting cell, thus promoting the binding of the transporter and the targeting cell, and increasing an efficiency of targeting the mRNA of the nanobody carried by the transporter to the targeting cell. The composition system can be used to preferentially target the mRNA of the nanobody to a variety of targeting cells, and suitable targeting cells comprise but are not limited to: liver cells, epithelial cells, endothelial cells, lung cells, bone cells, stem cells, mesenchymal cells, nerve cells, myocardial cells, adipocytes, immune cells, ovarian cells, testicular cells, tumor cells, etc.

The method described by the present invention and the tool system can be used for regulating the functions of various key functional proteins in cells, comprising various functional proteins related to disease occurrence, and the preferred solution is that the nanobody encoded by a mRNA is expressed in a cell and bound to a targeting protein. In some situations, the targeting protein is stabilized in inactivated conformation by the nanobody, and the function is inhibited, thus being used for treating diseases caused by over-activation of the targeting protein; and in other situations, the targeting protein is stabilized in activated conformation by the nanobody, and the function is activated, thus being used for treating diseases caused by insufficient activity of the targeting protein. Optional targeting proteins comprise but are not limited to: cell signaling pathway molecules, transcription factors, protein modifying enzymes (methylation, alkylation, acetylation, phosphorylation, ubiquitination, etc.), protein de-modification enzymes (demethylation, dealkylation, deacetylation, dephosphorylation, ubiquitination, etc.), nucleic acid modification enzymes (methylation and phosphorothiylation), nucleic acid de-modification enzymes (demethylation and dephosphorylation), structural proteins, important functional complexes, and other important disease marker proteins, etc. Diseases that can be controlled or treated by the method comprise but are not limited to: dysplasia, metabolic related diseases, tumors and cancers, autoimmune diseases, infectious diseases, autoimmune diseases, muscular and motor dysfunction, etc.

The nanobody encoded by a mRNA is used to target a list of potential intracellular targeting proteins (antibodies of targeting mutant proteins, wherein harmful proteins shall be selected to the greatest extent, a relationship between functional regulation and diseases, and localization of the development direction):

Various E3 ligases comprise:

AMFR, APC/Cdc20, Apc/Cdh1, C6orf157, Cbl, CBLL1, CHFR,CHIP, DTL (Cdt2), E6-AP, HACE1, HECTD1, HECTD2, HECDT3, HECW1, HECW2, HERC2, HECR3, HECR4, HECRS, HUWE1, HYD, ITCH, LNX1, mathogunin, MARCH-I, MARCH-II, MARCH-Ill, MARCH-IV, MARCH-V, MARCH-VI, MARCH-VII, MARCH-VIII, MARCH-X, MDM2, MEKK1, MIB1, MIB2, MycBP2, NEDD4, NEDD4L, Parkin, PELI1, Pirh2, PJA1, PJA2, RFFL, RFWD2, Rictor, RNFS, RNF8, RNF19, RNF190, RNF20, RNF34, RNF40, RNF125, RNF128, RNF38, RNF68, SCF/beta-Trcp, SCF/FBW7, SCF/Skp2, SHPRH, SIAH1, SIAH2, SMURF1, SMURF2, TOPORS, TRAF6, TRAF7, TRIM63, UBE3B, UBE3C, UBR1, UBR2, UHRF2, VHL, WWP1, WWP2, ZNRF1. Et al.

Various HDACs comprise:
HDAC1—HDAC11
Sirtuin 1—Sirtuin7

Various HATs comprise:
GCN5
PCAF
Tip60
MORF
MOZ
HBO1
P300
CBP
SRC-1
ACTR (RAC3, A1131, TRAM-1)
TIF-2 (GRIP1)
SRC-3
TFIIIC (p220, p110, p90)
CLOCK

Various methyltransferases comprise:
histone methyltransferase
DNA/RNA methyltransferase
N-terminal methyltransferase
Non-SAM Dependent Methyltransferases

Various demethyltransferases comprise:
KDM1 family
KDM2 family
KDM3 family
KDM4 family
KDM5 family
KDM6 family
protein-glutamate methylesterase

Various disease-related signaling pathway proteins comprise:
p53
KRAS
BRAF
BCR/ABL
BCL1
BCL2
PML-RARa
c-KIT
EGFR
EGFR (FISH)
HER2/neu (FISH)
JAK2
ALK (FISH)
DPD/TYMS
TPMT

Various infectious virus marker proteins or pathogen proteins comprise:
Composition of RSV (respiratory syncytial virus)

In the present invention, the regulation effect of the nanobody encoded by a mRNA on HDAC6-WT is particularly disclosed, and in the application, the nucleotide sequence of the mRNA is corresponding to a nucleotide sequence of HDAC6-CAT1, and the expressed nanobody is bound to HDAC6-WT.

Specifically, the nanobody has a VHH chain of an amino acid sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16, and has an encoding nucleotide sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

A template DNA molecule generating the mRNA is further comprised, and the nucleotide sequence is shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, which can be used to encode the nanobody for HDAC6-CAT1.

Further, in the application, we further disclose an expression vector which contains the nucleotide sequence of the DNA molecule above.

Moreover, further, a host cell is further comprised, and the host cell can express the nanobody for HDAC6-CAT1.

The present invention discloses a method that the nanobody encoded by a mRNA can efficiently and safely regulate and control the function of the specific intracellular protein. According to the method, the nanobody is encoded by the mRNA transcribed in vitro and having a modified base and a translational element, and a RNA transcription product is introduced into a specific cell through a physical method, so that the nanobody which can be bound to a specific targeting protein can be translated and expressed in cytoplasm.

Meanwhile, compared with the method using the mRNA as a vector to express a full length of a conventional IgG antibody or a fragment of the IgG antibody in a cell, since the nanobody is a single-chain polypeptide structure with small molecular weight (only 15 kd), the nanobody is very suitable to encode and express with a mRNA chain transcribed in vitro or chemically synthesized, and the difficulty of expressing the nanobody with the mRNA is far less than that of expressing a conventional multimeric IgG antibody with the mRNA. In addition, compared with the conventional IgG antibody or the fragment of the IgG antibody, the specificity and stability of the nanobody in recognizing a targeting protein are significantly improved, and the reliability of the nanobody in scientific research and clinical application is very good. More importantly, different nanobodies can activate or inhibit the function of the targeting protein according to different binding modes with the targeting protein, thus realizing bidirectional regulation of the protein function. Specifically, due to a small size, the nanobody can be inserted into a sunken space in a surface of the targeting protein, and if a conformational change induced by the binding leads to a more open active center of the targeting protein, the function of the targeting protein is more active; and on the contrary, if the binding causes the active center of the targeting protein to be shielded or distorted, the function of the protein is inhibited. To sum up, using the nanobody encoded by a mRNA as a tool to regulate the function of the intracellular protein has the characteristics of easy operation, stable performance and controllable effect, with a good clinical application prospect.

The present invention “using the nanobody encoded by a mRNA to regulate the function of the intracellular targeting protein” discloses a method capable of efficiently and safely regulating and controlling the function of the specific intracellular protein. According to the method, the nanobody is encoded by the mRNA transcribed in vitro and having the modified base and the translation element, and the RNA transcription product is introduced into the specific cell through the physical method, so that the nanobody which can be bound to the specific targeting protein can be translated and expressed in cytoplasm; and according to the different binding modes of the nanobody and the targeting protein, the method can be used for inhibiting the activity of the targeting protein and promoting the function of the targeting protein. By adopting the method, functions of disease-related proteins in cells can be effectively and specifically interfered with, thus achieving the object of disease treatment. Most disease-related proteins studied by predecessors are located in cells, and the present invention solves the problem that the nanobody targeting the intracellular protein cannot be used for clinical disease treatment, and fundamentally widens the applicable range of nanobody drugs. In addition, different from a conventional strategy for expressing the nanobody in a cell by using a DNA or a virus as a vector, the method eliminates a risk of changing a genome sequence of a host cell, has the features of safety and easy removal, and is highly suitable for clinical drug designation and production requirements.

### BRIEF DESCRIPTION OF THE DRAWINGS

FIG. 1 is a double enzyme digestion identification diagram of a recombinant plasmid, wherein 1 refers to a standard molecular weight of a DL2000 DNA; and 2 refers to a double enzyme digestion product of a pET32a-CAT1-JD recombinant plasmid, indicating that a HDAC6-CAT1-JD target fragment is successfully cloned into a pET32a (+) vector.

FIG. 2 shows SDS-PAGE analysis of a recombinant protein induction product, wherein M refers to a standard protein sample; and 1 refers to an inclusion body purification product of a BL21/CAT1-JD bacterium induction product, with a size of 48 kDa, indicating that a HDAC6-CAT1-JD recombinant protein is successfully expressed and has a good purification effect.

FIG. 3 is an ELISA antibody level detection chart of an immunized camel, wherein a serum antibody titer reaches 10⁴ after immunization, indicating that the HDAC6-CAT1-JD recombinant protein has a good immune effect.

FIG. 4 is a diagram showing expression and purification of a specific nanobody of a CAT1-JD recombinant protein in a host bacteria Colibacillus, wherein two nanobodies SEQ ID NO:4 and SEQ ID NO:6 are not successfully purified, and the remaining 14 nanobodies are well purified with a size of 15 kDa.

FIG. 5 is an ELISA diagram showing an affinity effect of a selected clone with HDAC6-WT, wherein 9 nanobodies have better reactions with the HDAC6-WT, which are respectively SEQ ID NO:2, SEQ ID NO:3, SEQ ID NO:5, SEQ ID NO:8, SEQ ID NO:11, SEQ ID NO:12, SEQ ID NO:14, SEQ ID NO:15, and SEQ ID NO:16.

FIG. 6 shows expression of a nanobody for a CAT1-JD recombinant protein in an eukaryotic host, wherein 16 nanobodies are all expressed in the eukaryotic host.

FIG. 7 is a Western-blot diagram showing an effect on a substrate acetylation level after expression of a Hela cell, wherein after a nanobody SEQ ID NO:5 is expressed in a eukaryote, the substrate acetylation level is increased, with an effect of a HDAC6 deacetylase inhibitor.

FIG. 8 is an IF diagram showing an effect on the substrate acetylation level after expression of a CHO-K1 cell, wherein red light of a cell transfected with the eukaryotic recombinant plasmid of SEQ ID NO:5 is more obvious than that of a cell not transfected with the recombinant plasmid, indicating that after a nanobody SEQ ID NO:5 is expressed in the eukaryotic cell, the substrate acetylation level is increased, so that the effect of the HDAC6 deacetylase inhibitor is proved once again.

FIG. 9 is a diagram showing an in vitro transcription method and an effect of each step, wherein 1 refers to a PCR product; 2 refers to mRNAs under capping incubation for 0.5 h; 3 refers to mRNAs under capping incubation for 1.5 h; 4 refers to a mRNA treated with Dnase 1; and 5 refers to a mRNA added with a tail poly(A), indicating that in vitro mRNA transcription has a better effect.

FIG. 10 is an EGFP diagram of in vivo binding of an anti-EGFP antibody of an in vitro transcription product, wherein an anti-actin antibody containing a GFP tag emits green fluorescence, an anti-GFP nanobody containing a His tag emits red fluorescence, and the green fluorescence and the red fluorescence are coincident, indicating that a mRNA of an anti-GFP nanobody transcribed in vitro is successfully transfected in a cell, which replaces an effect of a plasmid-transfected cell, can correctly recognize a GFP protein, and plays a certain biological function.

FIG. 11 is an EGFP diagram of in vivo binding of an anti-EGFP antibody of an in vitro transcription product, wherein an anti-golgi antibody containing a GFP tag emits green fluorescence, an anti-GFP nanobody containing a mCherry emits red fluorescence, and the green fluorescence and the red fluorescence are coincident, indicating that a mRNA of an anti-GFP nanobody transcribed in vitro is successfully transfected in a cell, which replaces an effect of a plasmid-transfected cell, can correctly recognize a GFP protein, and plays a certain biological function.

FIG. 12 is an vimentin diagram of in vivo binding of an anti-vimentin antibody of an in vitro transcription product, wherein an anti-vimentin antibody containing a mCherry emits red fluorescence, and the red fluorescence is in cell cytosol, indicating that a mRNA of an anti-vimentin nanobody transcribed in vitro is successfully transfected in a cell, which replaces an effect of a plasmid-transfected cell, can correctly recognize a vimentin protein, and plays a certain biological function.

FIGS. 13 to 14 are in vivo expression diagrams of an in vitro transcription product compared with a DNA, wherein a mRNA result is shown in FIG. 11 and a DNA result is shown in FIG. 12, indicating that the mRNA starts to be expressed 3 h after transcription, while the DNA starts to be expressed 24 h after transcription, and an expression time of the mRNA is earlier than that of the DNA.

FIG. 15 is a diagram showing an acetylation change after transferring a mRNA into a cell, wherein the mRNA of SEQ ID NO:5 is successfully expressed in the Hela cell, and a Tubulin acetylation level of a cell transfected with the mRNA of SEQ ID NO:5 is increased, indicating that the mRNA can replace the DNA and plays a role of the HDAC6 deacetylase inhibitor.

### DETAILED DESCRIPTION

In order to better understand the present invention, we further explain the invention below with reference to detailed embodiments and drawings, but it is worth noting because the embodiments of the invention are not limited to the detailed embodiments.

Reagents and raw materials used in the invention are commercially available or can be prepared according to literature methods. The test methods for specific conditions not specified in the embodiments of the invention are in accordance with conventional conditions or conditions suggested by manufacturers.

The application method of the present invention is introduced by taking a nanobody against an intracytoplasmic deacetylase HDAC6 as an example.

### Embodiment 1: Expression and Purification of HDAC6-CAT1 Truncated Protein

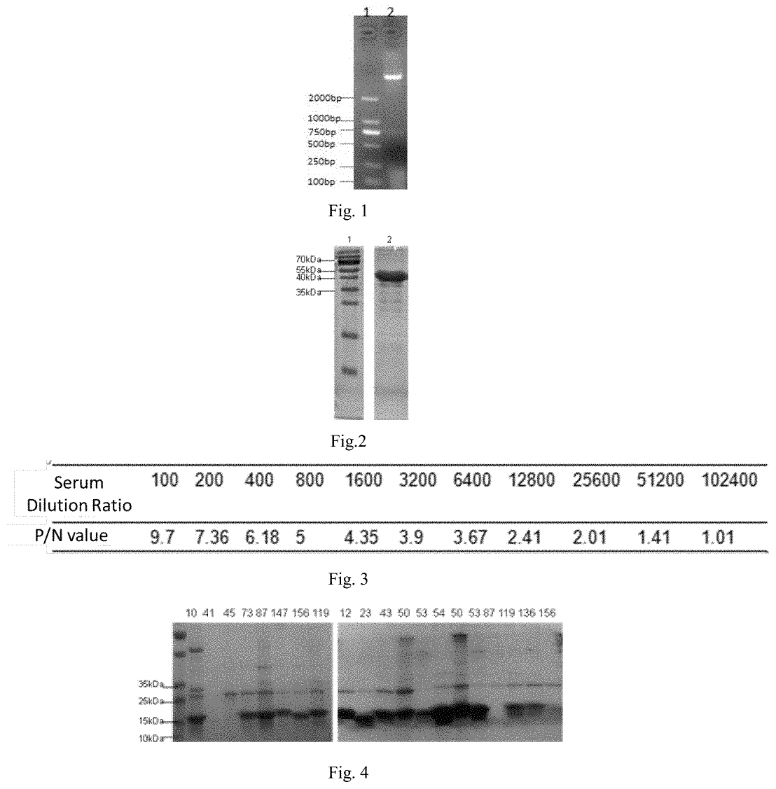

(1) According to a gene sequence of HDAC6, PCR primers: CAT1-JD-5-sal1 (cgaGTCGACgagcagttaaatgaattccattg) and CAT1-JD-3-not2 (gcgGCGGCCGCggcggccatctcacccttggggtcc) were designed by Premier Primer5.0 software, and an amplified gene fragment had a length of 801 bp; and (2) CAT1-JD-5-sall and CAT1-JD-3-not1 were used as the primers, and a HDAC6-WT recombinant plasmid was used as a template, so that 6-272 amino acids of HDAC6-Cat1 were amplified. A PCR reaction system was 25 μL, containing 12.5 μL of 2×PCR Mix (containing an enzyme), 1 μL of forward primer, 1 μL of reverse primer, 1 μL of DNA template, and 9.5 μL of double distilled water. Cycle parameters of PCR were: pre-degenerating at 95° C. for 8 min; degenerating at 95° C. for 40 s, annealing at 57° C. for 40 s, extending at 72° C. for 50 s, and 35 cycles; and finally, extending at 72° C. for 10 min. Observation was performed by 1% agarose gel electrophoresis. (3) A pET32a-Cat1-JD recombinant plasmid was reconstructed by a method as follows: gene fragments amplified by PCR were recovered by a commercially available DNA gel recovery kit, 10 μL of gene fragments purified and 10 μL of pET-32a (+) vectors were respectively subjected to double enzyme digestion with Sal I and Xho I restriction enzymes, and an enzyme digestion reaction could be performed in an EP tube of 0.5 mL with a water bath at 37° C. for 4 h to 5 h. The gene fragments and the vectors were recovered and purified after enzyme digestion. 7 μL of gene fragments after enzyme digestion and recovery were respectively connected with 1 μL of vectors after enzyme digestion by 1 μL of T4 ligases, and the connecting system was placed at 16° C. for 6 h to 8 h or overnight to obtain a connecting product. A DH5a competent cell was prepared and the connecting product was transformed, a plurality of dispersed single colonies were selected according to the growth of colonies on the plate above, inoculated into 3 mL of LB liquid medium containing 50 μg/mL ampicillin, and cultured overnight at 37° C. and 200 rpm. Under an aseptic condition, 1 μL of corresponding bacterial solution was taken as a template for PCR amplification, and after PCR, a PCR product was identified by 1.0% agarose gel electrophoresis. The bacterial solution corresponding to a sample with correct and specific PCR result was selected, and a recombinant plasmid was extracted using a plasmid extraction kit. (4) 1 μg of recombinant plasmids were respectively subjected to double enzyme digestion with 0.5 μL of Sal I and 0.5 μL of Not I restriction enzymes, and the recombinant plasmids which were identified to be corrected by double enzyme digestion were sent to Shanghai Invitrogen Company for sequencing. An enzyme digestion reaction could be performed in the EP tube of 0.5 mL, and an enzyme digestion process was the water bath at 37° C. for 4 h. After the completion of enzyme digestion, 10 μL of product after enzyme digestion was taken and subjected to 1% agarose gel electrophoresis, a result was shown in FIG. 1, it could be seen from the drawing that a CAT1-JD fragment obtained by enzyme digestion was consistent with an expected size, which was 801 bp, and the sequencing was completely correct, indicating that a HDAC6-CAT1-JD targeting fragment was successfully cloned into the pET32a (+) vector. (5) A recombinant plasmid pET32a-CAT1-JD with correct sequences was transformed into BL21 competent bacteria, and the obtained bacteria were named BL/CAT1-JD respectively. Single colonies of different recombinant bacteria were respectively taken into 3 mL of LB liquid culture medium containing kanamycin (with a content of 50 μg/mL), shaken at 37° C. for 2 h to 3 h, and IPTG (isopropyl thiogalactoside) with a final concentration of 1.0 mM was added when a bacterial liquid OD600 reached about 0.8 to 1.0, and shaking culture was continued for 5 h. After the completion of culture, centrifugation was performed at 8000 rpm for 3 min, bacteria were collected, and washed twice with a PBS solution, the bacteria were finally resuspended with 500 μL of PBS, ultrasonic crushing was performed with parameters of 200 W power and 10 min time (working for 3 s with an interval of 7 s), then centrifugation was performed at 8000 rpm for 5 min, a supernatant was collected, and a precipitate was resuspended with 500 μL of PBS at the same time. (6) 100 μL of supernatant and precipitate obtained by inducing expression in 1.7 were taken respectively, 25 μL of 5 x Loading Buffer was added, a sample was boiled in a water bath at 100° C. for 8 min, and then the mixture was added to sample wells in prepared polyacrylamide gel, with 20 μL per well. During electrophoresis, a concentrated gel run across at 80 V and a separated gel run out at 120 V. Then the gel was dyed with a Coomassie brilliant blue staining solution for 3 h, and a decolorizing solution was changed for 3 times to 5 times, with 1 hour each time, until a background color was decolorized completely, and photographing record was performed. A SDS-PAGE result was shown in FIG. 2. It could be seen from the figure that a lane 2 was an inclusion body purification product of a BL21/CAT1-JD bacterium induction product, with a size of 48 kDa, indicating that a HDAC6-CAT1-JD recombinant protein was successfully expressed with a good purification effect.

### Embodiment 2: Library Construction for HDAC6-CAT1 Nanobody

(1) 1 mg of CAT1-JD recombinant protein antigen and a Freund's adjuvant were mixed in equal volume, a Xinjiang two-humped camel was immunized once a week for a total of seven consecutive times of immunization, and during an immunization process, a B cell was stimulated to express a specific nanobody; (2) after seven consecutive times of immunization, 100 ml of peripheral blood lymphocytes of the camel were extracted, ELISA antibody level detection was performed to the immunized camel, a result was shown in FIG. 3, and a serum titer after immunization reached 10⁴, indicating that the HDAC6-CAT1-JD recombinant protein had a good immune effect; and a total RNA was extracted; (3) a cDNA was synthesized, and V_{H}H was amplified by nested PCR; (4) 20 ug of pMECS phage display vector and 10 ug of V_{H}H were digested with restriction enzymes Pst I and Not I and two fragments were connected; and (5) a connecting product was transformed into an electrically transformed competent cell TG1, a display library for a nanobody phage of a human antibody Fc fragment was constructed and a storage capacity was measured, wherein the storage capacity was about 1.2×10⁸.

### Embodiment 3: Screening Process of Nanobody for CAT1-JD Recombinant Protein

(1) 200 uL of recombinant TG1 cells were taken into a 2×TY culture medium for culture, 40 uL of auxiliary phages VCSM13 were added to infect the TG1 cells during the period, the phages were amplified by culturing overnight, the phages were precipitated with PEG/NaCl the next day, and the amplified phages were collected centrifugally; (2) 200 ug of CAT1-JD recombinant proteins dissolved in 100 mM of NaHCO3 with pH 8.2 were coupled on an ELISA plate, and left overnight at 4° C., while a negative control was set up; (3) 100 ul of 3% BSA was added the next day and sealed at a room temperature for 2 h; (4) after 2 h, 100 ul of amplified phages (2×10¹¹ tfu immunized camel nanobody phage display gene pool) were added and reacted at a room temperature for 1 h; (5) washing was performed five times with PBS +0.05% Tween −20 to wash away the bound phages; (6) the phage specifically bound to the human antibody Fc fragment was dissociated by trypsin with a final concentration of 25 mg/ml, a TG1 cell of Colibacillus in a logarithmic growth phase was infected, and cultured at 37° C. for 1 h, the phage was generated and collected for the next round of screening, and the same screening process was repeated for 3 rounds to be gradually enriched.

### Embodiment 4: Screening of Specific Positive Clones by Enzyme-Linked Immunosorbent Assay (ELISA) of Phage

(1) 175 single colonies were selected from the cell culture plate after the three rounds of screening above and respectively inoculated into a deep 96-well plate of a TB culture medium containing 100 ug/mL ampicillin, a blank control was set, after culturing at 37° C. to a logarithmic phase, IPTG with a final concentration of 1 mM was added, and cultured overnight at 28° C.; (2) a crudely extracted antibody was obtained by an osmotic bursting method, and the antibody was transferred to an antigen-coated ELISA plate, and placed at a room temperature for 1 h; (3) an unbound antibody was washed off with PBST, and 100 ul of Mouse anti-HA tag antibody (purchased from Covance) diluted by 1:2000 was added, and placed at a room temperature for 1 h; (4) the unbound antibody was washed off with PBST, and 100 ul of Anti-mouse alkaline phosphatase conjugate (purchased from Sigma) was added, and placed at a room temperature for 1; (5) the unbound antibody was washed off with PBST, an alkaline phosphatase color developing solution was added, and after reaction for 5 min to 10 min, an absorption value was read at OD_{405 nm} wavelength on a microplate reader; (6) when an OD value of a sample well was larger than 5 times that of a control well, a positive clone well was determined; and (7) bacteria in the positive clone well were rotated and shaken to the LB medium containing 100 ug/ul ampicillin to extract plasmids and sequence the plasmids.

According to sequence alignment software Vector NTI, gene sequences of cloned strains were analyzed, and the strains with the same sequences of FR1, FR2, FR3, FR4, CDR1, CDR2 and CDR3 were regarded as the same cloned strain, while the strains with different sequences were regarded as different cloned strains, and finally 16 specific nanobodies of CAT1-JD recombinant protein were obtained. An amino acid sequence of the antibody was a FR1-CDR1-FR2-CDR2-FR3-CDR3-FR4 region shown as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, forming the entire V_{H}H.

### Embodiment 5: Expression, Purification and Identification of Specific Nanobody of CAT1-JD Recombinant Protein in Host Bacterium Colibacillus

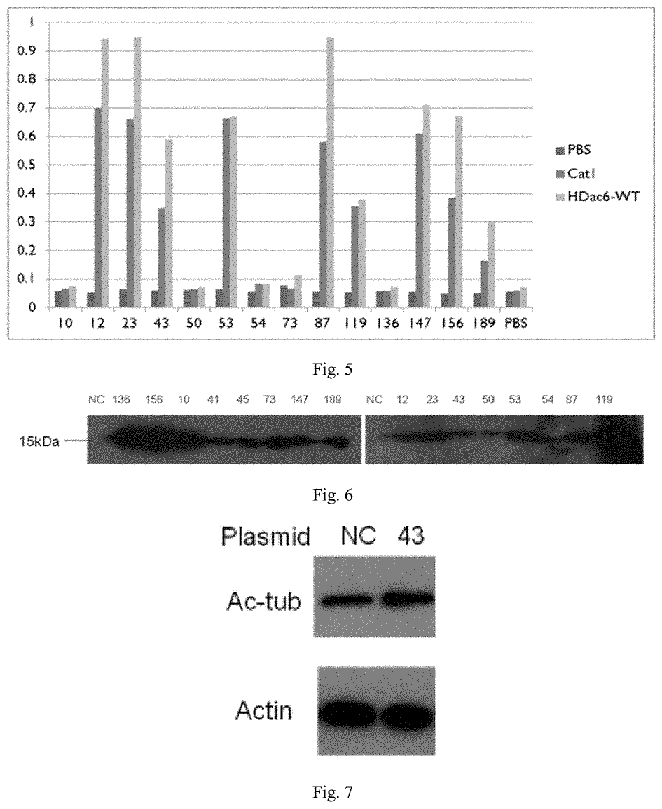

(1) Plasmid points of different cloned strains obtained by sequencing analysis were transformed into colibacillus WK6, and the mixture was coated on a LB +amp +glucose, i.e., a culture plate containing ampicillin and glucose, and cultured overnight at 37° C.; (2) single colonies were selected to be inoculated in 5 ml of LB culture solution containing ampicillin, and subjected to shake cultivation at 37° C. overnight; (3) 1 mL of strains cultured overnight were inoculated into 330 mL of TB culture solution, and subjected to shake cultivation at 37° C. overnight, when an OD_{600 nm} value reached 0.6 to 0.9, 1 M of IPTG was added, and subjected to shake cultivation at 28° C. overnight; (4) centrifugation was performed to collect the colibacillus, and a crude extract of the antibody was obtained by an osmotic bursting method; (5) the antibody was purified by nickel column affinity chromatography to obtain a high-purity nanobody, as shown in FIG. 4, two nanobodies SEQ ID NO:4 and SEQ ID NO:6 were not successfully purified, and the remaining 14 nanobodies were well purified, with a size of 15 kDa; (6) the antibody was transferred to an ELISA plate coated with HDAC6-WT, and was placed at a room temperature for 1 h; (7) an unbound antibody was washed off with PBST, and 100 ul of Mouse anti-HA tag antibody (purchased from Covance) diluted by 1 : 2000 was added, and placed at a room temperature for 1 h; (8) the unbound antibody was washed off with PBST, and 100 ul of Anti-mouse alkaline phosphatase conjugate (purchased from Sigma) was added, and placed at a room temperature for 1; (9) the unbound antibody was washed off with PBST, an alkaline phosphatase color developing solution was added, after reaction for 5 min to 10 min, an absorption value was read at OD_{405 nm} wavelength on a microplate reader, and a result was shown in FIG. 5, wherein 9 nanobodies had better reaction with HDAC6-WT, which were respectively SEQ ID NO:2, SEQ ID NO:3, SEQ ID NO:5, SEQ ID NO:8, SEQ ID NO:11, SEQ ID NO:12, SEQ ID NO:14, SEQ ID NO:15 and SEQ ID NO:16.

### Embodiment 6: Expression of Nanobody for CAT1-JD Recombinant Protein in Eukaryotic Host and Effect on Substrate Acetylation Level After In Vivo Expression

(1) plasmids of different cloned strains obtained by sequencing analysis above were connected in series with EGFP to construct an eukaryotic recombinant plasmid pcDNA.1, the eukaryotic recombinant plasmid and a pcDNA3.1 empty plasmid were transfected into a Hela cell by a liposome 2000, the cell was lysed after 36 h, Western-blot identification was performed, a protein was transferred to a NC membrane, and 10 mL of Mouse anti-His Tag antibody (purchased from Sigma) diluted by 1:2000 was added, and placed at a room temperature for 1 h; an unbound antibody was washed off with PBST, 10 mL of Goat Anti-rabbit HRP conjugate (purchased from Sigma) diluted by 1:2000 was added, and placed at a room temperature for 1 h; the unbound antibody was washed off with PBST, and exposed, as shown in FIG. 6, all 16 nanobodies were expressed in eukaryotic hosts; (2) Western-blot identification was performed on the cell lysate in (1), the protein was transferred to the NC membrane, and 10 mL of Rabbit anti-Tubulin K40 antibody (purchased from Abcam) diluted by 1:2000 was added, and placed at a room temperature for 1 h; the unbound antibody was washed off with PBST, and 10 mL of Goat Anti-rabbit HRP conjugate (purchased from Sigma) diluted by 1:2000 was added, and placed at a room temperature for 1 h; the unbound antibody was washed off with PBST, and exposed, as shown in FIG. 7, after a nanobody SEQ ID NO:5 was expressed in a eukaryote, a substrate acetylation level was increased, and the effect of a HDAC6 deacetylase inhibitor was achieved; (3) the eukaryotic recombinant plasmid of SEQ ID NO:5 was transfected into a CHO-K1 cell, indirect immunofluorescence was performed after 36 h, a cell culture solution was washed off with PBST, and after fixing the cell, 100 μL of Rabbit anti-Tubulin K40 antibody (purchased from Abcam) diluted by 1:2000 was added, and placed at a room temperature for 1 h; the unbound antibody was washed off with PBST, and 100 μL of Goat Anti-rabbit A568 conjugate (purchased from Sigma) diluted by 1:2000 was added, and placed at a room temperature for 1 h; the unbound antibody was washed off with PBST, and observed with a fluorescence microscope, a result was shown in FIG. 8, red light of a cell transfected with the eukaryotic recombinant plasmid of SEQ ID NO:5 was more obvious than that of a cell not transfected with the recombinant plasmid, indicating that after a nanobody SEQ ID NO:5 was expressed in the eukaryote, the substrate acetylation level was increased, so that the effect of the HDAC6 deacetylase inhibitor was proved once again.

### Embodiment 7: In Vitro Transcription Method and Diagram Showing Effects of Each Step

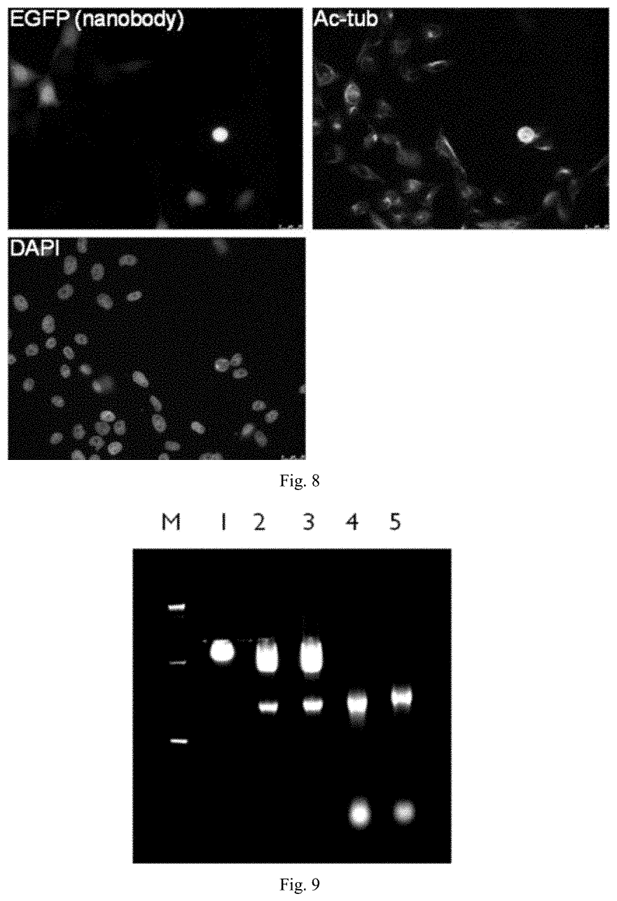

In vitro transcription PCR primers: T7-5UTR-GFP-V_{H}H-pcDNA3.1-F (TAATACGACTCACTATAGGGGAGACCCAAGCTGGCTA) and 3URT-GFP-V_{H}H-pcDNA3.1-R (AGAATAGAATGACACCTACTC) were designed, comprising a T7 promoter, and 5′UTR and 3′UTR regions, a recombinant pcDNA3,1 plasmid of an anti-EGFP nanobody was used as a template, and a large number of DNA templates were obtained by PCR; 50 μL of PCR reaction system was recovered by NaAc, and transferred to a new PCR tube; a DNA was transcribed into a mRNA by HiScribe™ T7 ARCA mRNA Kit (with Tailing) (mRNA in vitro transcription kit, purchased from NEB Company), as shown in FIG. 9, 1 referred to a PCR product; 2 referred to mRNAs under capping incubation for 0.5 h; 3 referred to mRNAs under capping incubation for 1.5 h; 4 referred to a mRNA treated with Dnase 1; and 5 referred to a mRNA added with a tail poly(A), indicating that in vitro mRNA transcription had a better effect.

### Embodiment 8: In Vivo Expression Diagram of In Vitro Transcription Product

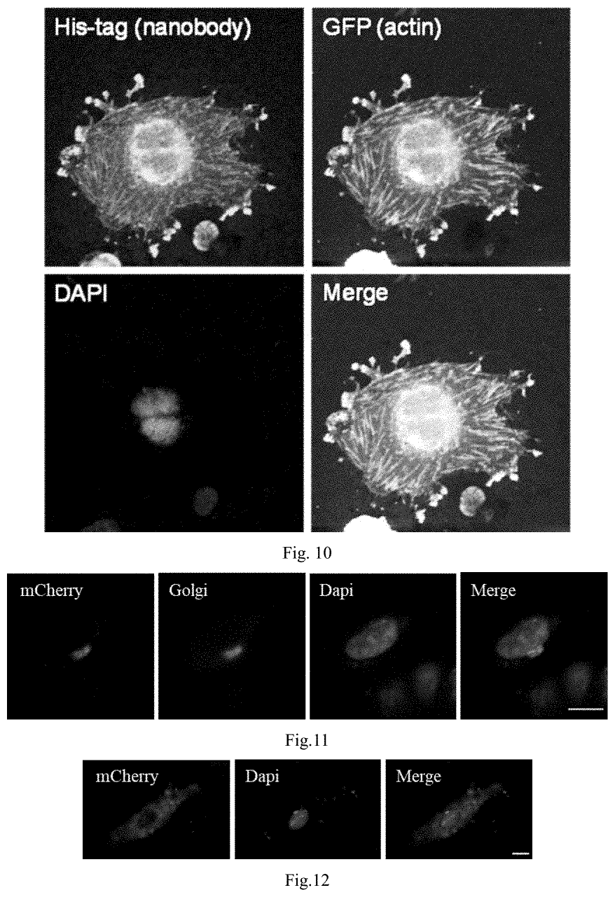

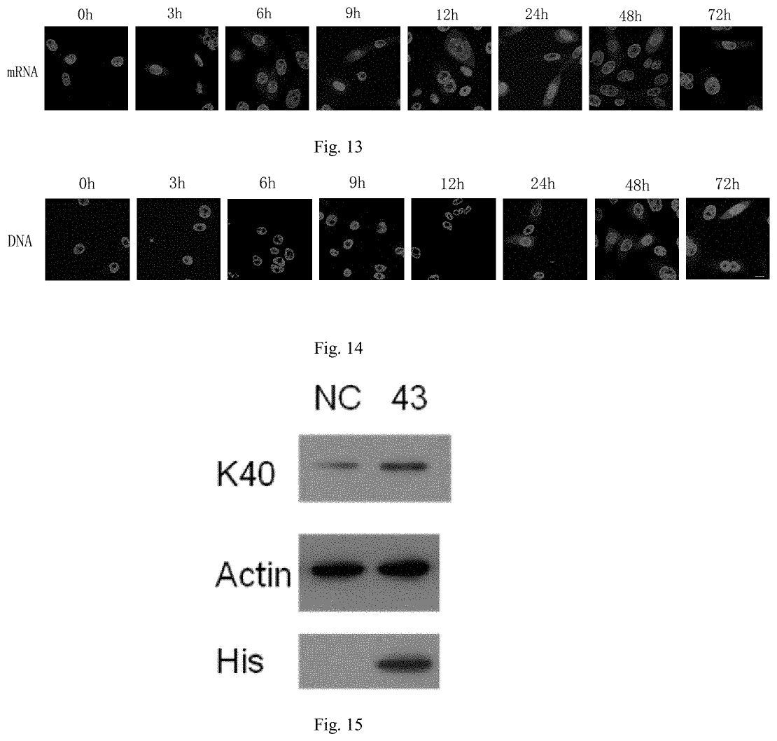

(1) 15 ug of mRNA transcribed in vitro was added with 8 uL of lipo2000 to be dissolved in 250 uL of DMEM, a CHO-K1 cell stably expressing an anti-actin antibody containing a GFP tag, as well as an anti-golgi antibody containing a GFP tag, were transfected, indirect immunofluorescence was performed after 36 h, a cell culture solution was washed off with PBST, after fixing the cell, 100 μL of Mouse anti-His antibody (purchased from Sigma) diluted by 1:2000 was added, and placed at a room temperature for 1 h; an unbound antibody was washed off with PBST, 100 μL of Goat Anti-mouse A568 conjugate (purchased from Sigma) diluted by 1:2000 was added, and placed at a room temperature for 1 h; the unbound antibody was washed off with PBST, and observed with a fluorescence microscope, as shown in FIG. 10, the anti-actin antibody containing the GFP tag emitted green fluorescence, and an anti-GFP nanobody containing a His tag emitted red fluorescence, and the green fluorescence and the red fluorescence were coincident, indicating that a mRNA of an anti-GFP nanobody transcribed in vitro was successfully transfected in a cell, which replaced an effect of a plasmid-transfected cell, could correctly recognize a GFP protein, and played a certain biological function.

(2) Meanwhile, cells were collected at different time points after transfection, and an occurrence time of a mRNA was observed, taking a DNA as a control. Taking 0 h, 3 h, 6 h, 9 h, 12 h, 24 h, 48 h and 72 h as time points respectively, cells were collected, indirect immunofluorescence was performed, a cell culture solution was washed off with PBST, after fixing the cell, 100 μL of Mouse anti-His antibody (purchased from Sigma) diluted by 1:2000 was added, and placed at a room temperature for 1 h; an unbound antibody was washed off with PBST, and 100 μL of Goat Anti-mouse A568 conjugate (purchased from Sigma) diluted by 1:2000 was added, and placed at a room temperature for 1 h; the unbound antibody was washed off with PBST, and observed with a fluorescence microscope, a mRNA result was shown in FIG. 11, and a DNA result was shown in FIG. 12, indicating that the mRNA started to be expressed 3 h after transcription, while the DNA started to be expressed 24 h after transcription, and an expression time of the mRNA was earlier than that of the DNA.

### Embodiment 9: Diagram Showing Acetylation Change After Transferring mRNA Into Cell

(1) The method was the same as that in the Embodiment 6, and a mRNA of SEQ ID NO:5 was transcribed in vitro using a recombinant pcDNA3.1 plasmid of SEQ ID NO:5 as a template. The method was the same as that in the Embodiment 5, and Western-blot identification was performed. A result was shown in FIG. 12, the mRNA of SEQ ID NO:5 was successfully expressed in the Hela cell, and a Tubulin acetylation level of a cell transfected with the mRNA of SEQ ID NO:5 was increased, indicating that the mRNA could replace the DNA and played a role of the HDAC6 deacetylase inhibitor.

The foregoing shows and describes the basic principles, main features, and advantages of the invention. Those skilled in the art should know that the invention is not limited by the embodiments above, and the descriptions in the embodiments and the specification above only describe the principle of the invention. The invention may also have various modifications and improvements without deviating from the spirit and the scope of the invention, and all these modifications and improvements shall fall within the scope of the invention sought to be protected.

What is claimed is:

1. A nanobody encoded by a mRNA, wherein the mRNA is translated into a single-chain nanobody that can specifically recognize a targeting protein in a cell.

2. The nanobody encoded by a mRNA according to claim 1, wherein a mRNA molecule carries at least one modification to enhance a stability of the mRNA molecule.

3. The nanobody encoded by a mRNA according to claim 1, wherein the mRNA molecule carries at least one modification to enhance a translational efficiency of the mRNA molecule.

4. The nanobody encoded by a mRNA according to claim 1, wherein the mRNA molecule carries at least one modification to reduce an immunogenicity of the mRNA molecule.

5. The nanobody encoded by a mRNA according to claim 1, wherein an upstream and a downstream of a mRNA-encoding protein are modified within intracellularly localizational signals.

6. A process of the nanobody encoded by a mRNA according to claim 1 is regulating a function of an intracellular target protein.

7. The process according to claim 6, wherein the mRNA encoding the nanobody enters a cell through a transporter.

8. The process according to claim 7, wherein the transporter is added with a ligand bound to a surface marker of a targeting cell to be regulated.

9. The process according to claim 6, wherein a nucleotide sequence of the mRNA is corresponding to a nucleotide sequence of HDAC6-CAT1, and the nanobody expressed by the mRNA is bound to HDAC6-Wildtype.

10. The process according to claim 9, wherein the nanobody has a V_{H}H chain of an amino acid sequence encoded by a nucleotide sequence that is selected from a group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

11. A method for rapidly delivering a nanobody into a cell by expressing a mRNA of a nanobody in the cell, comprising the following steps:
a) synthesizing one or more mRNA(s) which code for nanobodies;
b) purifying the mRNAs; optionally modifying the mRNAs;
c) transfecting the mRNAs into a cell;
d) preparing the nanobodies by translating the mRNAs into the nanobodies in the cell;
wherein the mRNA(s) is (are) one or more nucleotide sequence(s) selecting from a group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, or mRNA(s) is (are) anti-GFP nanobody, anti-vimentin nanobody.

12. The method of claim 11, wherein the one or more mRNAs encoding the nanobody are encapsulated within one or more liposomes.

13. The method of claim 12, wherein the one or more liposomes comprise one or more of cationic lipid, neutral lipid, cholesterol-based lipid, and PEG-modified lipid.

14. The method of claimll, the method comprising:
administering to a cell a first mRNA encoding a nanobody, and wherein the nanobody is produced by the cell.

15. The method of claim 14, wherein the cell is a mammalian cell.

16. The method of claim 14, wherein the cell is a human cell.

17. The method of any one of claims 14, wherein the cell is a cultured cell.

18. The method of any one of claims 14, wherein the nanobody is expressed intracellularly.
